(19) United States
(12) Patent Application Publication
(10) Pub. No.: US 2026/0256424 A1
(43) Pub. Date: Sep. 3, 2026

(54) SYSTEMS AND METHODS FOR PHYSIOLOGICAL PARAMETER MONITORING

(71) Applicant: Covidien LP, Mansfield, MA (US)

(72) Inventors: Clark R. Baker, JR., Newman, CA (US); Scott J. McGonigle, Loanhead (GB); James P. Ochs, Palo Alto, CA (US); Kelly N. Kneale, Denver, CO (US)

(73) Assignee: Covidien LP, Mansfield, MA (US)

(21) Appl. No.: 19/550,609

(22) Filed: Feb. 26, 2026

Related U.S. Application Data
(60) Provisional application No. 63/764,759, filed on Feb. 28, 2025.
(60) Provisional application No. 63/884,402, filed on Sep. 19, 2025.

(51) Int. Cl. A61B 5/00; A61B 5/08; A61B 5/1455; G16H 40/63

(52) U.S. Cl. CPC A61B 5/7203; A61B 5/08; A61B 5/14552; G16H 40/63

(57) ABSTRACT

The techniques of this disclosure generally relate to systems and methods for monitoring physiological parameters of a patient. A medical monitor operates in a first mode in which it measures a baseline value of a physiological parameter and a trend value of the physiological parameter. The medical monitor determines an offset between the baseline value and the trend value. In response to evaluating spectral data of the patient, the monitor then operates in a second mode in which it outputs one of the trend value or the trend value adjusted by the determined offset.

### CROSS-REFERENCE TO RELATED APPLICATIONS

This application claims the benefit of U.S. Application Ser. No. 63/884,402, filed Sep. 19, 2025, and U.S. Application Ser. No. 63/764,759, filed Feb. 28, 2025, the entire contents of both of which are incorporated by reference.

### FIELD

The present technology is generally related to systems and methods for monitoring physiological parameters and physiological parameter trends of a patient.

### BACKGROUND

In the field of medicine, healthcare providers often desire to monitor certain physiological characteristics of their patients. Monitoring devices have been developed for monitoring physiological parameters such as pulse rate, respiration rate, oxygen saturation, temperature, posture, and more. These devices provide healthcare providers with information they need to monitor the status of their patients and determine when medical intervention is necessary.

An example of a physiological parameter of a patient is arterial oxygen saturation measured by a pulse oximeter. A pulse oximeter uses optical signals to non-invasively measure the oxygen saturation in a patient's blood. Two wavelengths of light (typically red and infrared) are passed through the patient's tissue and detected by a sensor, producing light signals that vary in amplitude with the patient's pulse. The relative absorption of these light signals is used to determine the blood oxygen saturation (the ratio of oxygenated and deoxygenated blood), and the frequency of the pulses is used to determine pulse rate.

Another example of a physiological parameter of a patient is tissue or regional oximetry. Regional oximetry is a non-invasive measurement of oxygenation in tissue using near infrared light. Light in the red and infrared spectra are emitted into the tissue and detected by two or more detectors after the light passes through the tissue. The detectors are spaced apart from each other. The light reaching the closer detector (positioned closer to the emitter) has passed through more shallow tissue such as skin, bone, and dermal fat layers, and the light reaching the farther detector (positioned farther from the emitter) has passed through deeper tissue such as brain, kidney, or other organs. The shallow measurement is subtracted from the deeper measurement to focus on the deeper tissue of interest, and the ratio of light absorption between the red and infrared signals indicates the oxygenation of the tissue. In aspects, algorithms for determining oxygenation can be based on evaluating absorption of four wavelengths of light or two wavelengths of light. In further aspects, while algorithms based on four wavelengths of light may more accurately estimate the oxygenation, algorithms based on two wavelengths of light may be more responsive to changes in oxygenation. The present disclosure relates to identifying methods and systems for providing both accurate and responsive estimations of tissue oxygenation.

### SUMMARY

The techniques of this disclosure generally relate to systems and methods for monitoring physiological parameters of a patient.

In an embodiment, a method for medical monitoring of a patient includes receiving, from one or more sensors applied to the patient, a first physiological signal and a second physiological signal. The signals are responsive to a physiological parameter of the patient. The method includes determining, by one or more computer processors, a baseline value of the physiological parameter from the first physiological signal and a trend value of the physiological parameter from the second physiological signal. The method also includes outputting the baseline value during a baseline monitoring session, establishing an offset between the baseline value and the trend value during the baseline monitoring session, and storing the offset. The method also includes switching from the baseline monitoring session to a trend monitoring session and in response to the switching, adjusting the trend value by the offset. The method includes outputting the adjusted trend value of the physiological parameter during the trend monitoring session such as to a monitor for display or alarms.

In an embodiment, a method for monitoring a physiological parameter of a patient includes receiving a physiological signal from a sensor applied to a patient. The physiological signal includes a plurality of channels of data responsive to a physiological parameter of the patient. The method includes deriving a physiological parameter baseline from the plurality of channels, deriving a physiological parameter trend from a subset of the plurality of channels, and storing an offset between the physiological parameter baseline and the physiological parameter trend. The method includes producing a combined physiological parameter of the patient by adjusting the physiological parameter trend by the offset, and outputting the combined physiological parameter for monitoring or display.

In an embodiment, a method is provided for determining whether to apply an offset to a physiological parameter trend value for a patient. The method includes receiving optical data associated with the patient. The optical data is detected at a plurality of wavelengths over time. The method includes computing f-signal spectra values for each wavelength of the plurality of wavelengths over time, and applying first criteria to the computed f-signal spectra values. When the first criteria are met, the method includes applying second criteria to the computed f-signal spectra values. When the second criteria are not met, the method includes outputting the physiological parameter trend value.

In an embodiment, a method for monitoring a physiological parameter of a patient includes receiving a physiological signal from a sensor applied to a patient. The physiological signal includes a plurality of channels of data responsive to a physiological parameter of the patient. The method includes deriving a physiological parameter baseline from the plurality of channels, deriving a physiological parameter trend from a subset of the plurality of channels, and storing an offset between the physiological parameter baseline and the physiological parameter trend. The method also includes producing a combined physiological parameter of the patient by adjusting the physiological parameter trend by the offset, and outputting the combined physiological parameter for monitoring or display.

In an embodiment, a method for monitoring a physiologic parameter of a patient includes receiving, at a medical monitor, a physiological signal from a sensor applied to a patient. The physiological signal is responsive to a physiologic parameter of the patient. The method includes operating the medical monitor in a first measurement mode comprising. In the first mode, the method includes deriving a first estimate of the physiologic parameter of the patient, deriving a second estimate of the physiologic parameter of the patient, deriving an offset between the first estimate and the second estimate, outputting the first estimate to an alert or display, and evaluating a stability of the offset. Upon determining that the stability satisfies a criterion, the method includes switching to a second measurement mode. In the second mode, the method includes deriving the second estimate of the physiologic parameter of the patient, adjusting the second estimate by the offset, and outputting the adjusted second estimate to the alert or display. Subsequently, a change is detected in the stability, the second estimate, the physiological signal, or the sensor, and the method includes returning the medical monitor to the first measurement mode due to the change.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a front and schematic view of a system for medical monitoring of a patient, including sensors to be applied to a patient, according to an embodiment.

FIG. 2 is a chart depicting baseline values and trend values of a physiological parameter derived from the medical monitoring system of FIG. 1.

FIG. 3 is a flowchart depicting a method of monitoring a patient in baseline and trend monitoring sessions, according to an embodiment.

FIG. 4 is a flowchart depicting a method of deriving a physiological parameter of a patient, according to an embodiment.

FIG. 5 is a flowchart depicting a method of deriving a physiological parameter of a patient, according to an embodiment.

FIGS. 6A-6C are charts depicting plots of normalized f-signal spectra versus wavelength for a population of patients, according to an embodiment.

FIG. 7 is a flowchart depicting a method of determining whether to apply an offset when deriving a physiological parameter, according to an embodiment.

FIG. 8 is a flowchart depicting a method of determining a physiological parameter of a patient, according to an embodiment.

### DETAILED DESCRIPTION

Embodiments of the present disclosure provide systems and methods for monitoring physiological parameter baselines and physiological parameter trends of a patient. In embodiments, a medical monitor receives incoming signals from one or more sensors applied to the patient. These may be optical, electrical, motion, thermal, or other types of medical sensors described more fully below. The monitor derives physiological parameters of the patient from these signals, including a physiological parameter baseline and a physiological parameter trend. Examples include pulse rate, oxygen saturation, respiration rate, and other vital signs of the patient. In aspects, a “baseline” value represents an estimate of a physiological parameter that is derived from a first input signal or algorithm that is relatively more complex (compared to the second input signal or algorithm), while a “trend” value represents an estimate of a physiological parameter that is derived from a second input signal or algorithm that is relatively less complex (compared to the first input signal or algorithm). For example, the first input signal may be higher-fidelity, higher-resolution, higher quality, or higher power than the second input signal. The first input signal may include a higher sampling rate, a larger number of data channels, greater signal power or intensity, higher resolution, less filtering, or other differences as compared to the second input signal. Alternatively or in addition to those differences between the first and second input signals, the first algorithm used to derive the baseline value may be more complex than the second algorithm used to derive the trend value. For example, the first algorithm may use more processing power and time as compared to the second algorithm, and thus may be slower to provide the baseline value. The first algorithm may use a larger portion of an input signal than the second algorithm. The relatively rich information in the first input signal or used by the first algorithm enables the first method to derive the baseline value, which represents an absolute value of the physiological parameter for most patients. By comparison, the second input signal may be lower-fidelity, lower-resolution, lower quality, or lower power than the first input signal. The second input signal may include a lower sampling rate, a smaller number of data channels, lower signal power or intensity, lower resolution, more filtering, or other differences as compared to the first input signal. The second algorithm used to derive the trend value may use less processing power and less time as compared to the first algorithm, and thus may be faster to provide the trend value. The second algorithm may use a smaller portion or subset of the input signal than the first algorithm. The relatively more sparse (or less dense) information in the second input signal or used by the second algorithm enables the second method to derive the trend value, which trends with the physiological parameter of the patient but does not reflect an absolute value of a physiological parameter for most patients.

An example baseline algorithm utilizes input signals from four wavelengths, and an example trend algorithm utilizes input signals from two wavelengths. In some aspects, the baseline may be more numerically accurate, for most patients, than the trend; however, in other aspects, the trend may be more responsive to changes in the physiological parameter than the baseline. Accordingly, as described herein, the present disclosure proposes deriving an accurate estimate of a physiological parameter by using a combination of the baseline and the trend.

As used herein, a “true value” of a physiological parameter is a measured value of the physiological parameter that is obtained directly (e.g., from blood or tissue, sometimes referred to as the gold standard measurement) and an “absolute value” of a physiological parameter is defined as a derived value that is obtained indirectly (e.g., through skin) that is within an acceptable range of the true value of the physiological parameter. Using pulse oximetry as an example, a “true value” can be obtained from an arterial blood gas analysis, and an “absolute value” can be obtained from a pulse oximetry sensor sensing through the skin. As mentioned above, for most patients, a baseline value of a physiological parameter (e.g., derived using the four-wavelength algorithm) may be more numerically accurate than a trend value of the physiological parameter (e.g., derived using the two-wavelength algorithm). For example, for most patients, the baseline value corresponds to the absolute value of the physiological parameter. Additionally, for most patients, while the trend value is highly correlated (e.g., moves or changes) with the absolute value of a physiological parameter, the trend value is numerically offset or displaced or scaled from the absolute value. However, for some patients, the baseline value is high biased with respect to the absolute value of the physiological parameter and, for this group of patients, the trend value may more accurately represent the absolute value of the physiological parameter.

In an embodiment, the medical monitor operates in a first mode in which it derives a baseline value and a trend value of the physiological parameter and determines an offset between the baseline value and the trend value. The monitor then operates in a second mode in which it outputs the trend value adjusted by the determined offset, resulting in an adjusted trend value that is based on the trend value but also accurately reflects the absolute value of the physiological parameter. In the second mode, the monitor operates in a trend monitoring session in which the monitor derives the trend value from the incoming input signals, adjusts the trend value based on the offset, and outputs the adjusted trend value. As compared to the baseline value, the physiological parameter trend value can be determined from a subset of incoming input signals and/or may be more responsive to changes in the patient's physiological status and/or may be more effective in certain monitoring conditions. When the monitor detects that the offset between the baseline value and trend value has changed by a threshold amount, or if reset conditions exist such as sensor repositioning or noise in the incoming signals, the monitor returns to a baseline monitoring mode and re-establishes a new offset. As discussed more fully herein, the monitor derives both a baseline value and a trend value of the physiological parameter, provides outputs based on one or both of the baseline value and the trend value, and generates displays or alerts that are accurate, efficient, and responsive to changes in the patient's physiological status.

While regional oximetry (also referred to as tissue oximetry) is used as an example in many of the discussions and figures below, it should be understood that the systems and methods described herein may be applied to various different physiological signals and parameters derived from those signals. Examples of sensors include optical, electrical, acoustic, temperature, motion, touch, capacitive, proximity, impedance, and other sensors that produce signals responsive to physiological parameters of a patient. Examples of physiological parameters derived from these signals include regional/tissue oximetry (rSO2, tissue oxygen saturation, and/or perfusion), pulse oximetry (SpO2, arterial oxygen saturation, and/or pulse rate), hemoglobin concentration (oxygenated, deoxygenated, and/or total), heart rate and electrocardiogram (ECG) waveforms, depth of consciousness parameters, EEG (electroencephalogram), respiratory parameters (such as respiration rate, respiratory effort, tidal volume, and/or apnea), skin or core temperature, posture, activity, motion status, and others.

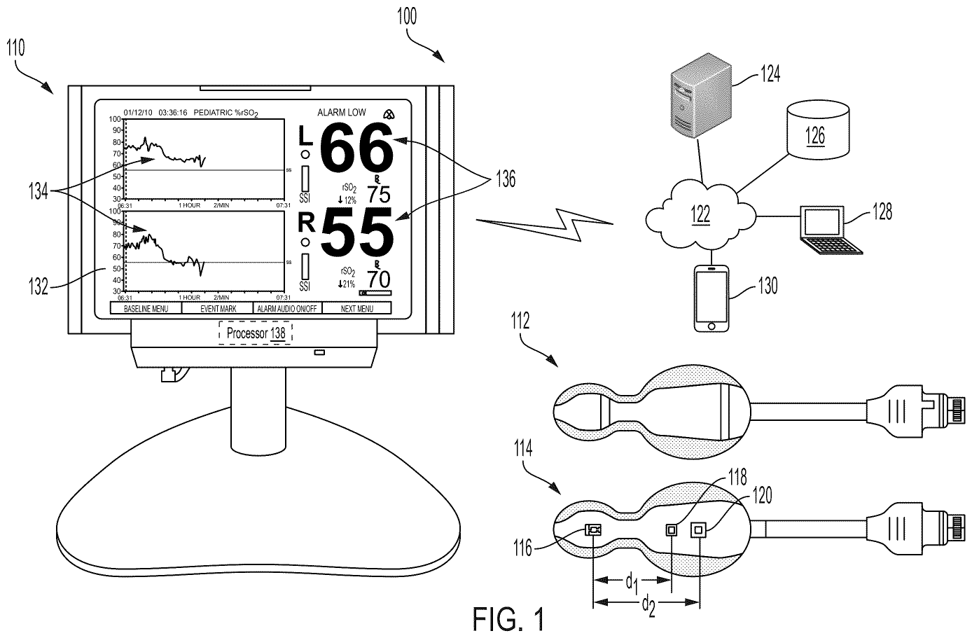

A system 100 (e.g., a medical monitoring system) according to an embodiment is shown in FIG. 1. In this embodiment, the system 100 measures tissue oximetry of a patient, such as measuring oxygenation of cerebral tissue, kidneys, or other organs. The system 100 includes a monitor 110 that connects through wired or wireless connections to a first regional oximetry sensor 112 and a second regional oximetry sensor 114. In aspects, the monitor 110 may be part of a computing device (not shown) or may comprise computing components (e.g., a processor 138) capable of processing, storing, receiving, and/or transmitting various signals received from the first regional oximetry sensor 112 and/or the second regional oximetry sensor 114. In use, the first regional oximetry sensor 112 and the second regional oximetry sensor 114 are placed in contact with a patient's skin and emit wavelengths of light into the patient's skin. The first regional oximetry sensor 112 is shown with the patient-contacting side facing down (not visible). The second regional oximetry sensor 114 is shown with the patient-contacting side facing up, showing the sensing components.

The sensing components include an emitter 116 that emits light at two or more wavelengths, and two detectors 118 and 120 (e.g., photodetectors) spaced apart from the emitter 116. The first detector 118 is spaced apart from the emitter 116 by a distance d1, and the second detector 120 is spaced apart from the emitter 116 by a second, longer distance d2. As a result, the light emitted by the emitter 116 travels through shallower tissue to reach the first detector 118 and deeper tissue to reach the second detector 120. The shallower signal is subtracted from the deeper signal so that the contributions from the patient's skin and other dermal layers are removed and the contributions from the patient's underlying tissue remain. In some embodiments, the emitter 116 emits four wavelengths of light, and the first detector 118 and the second detector 120 produce four channels of data representing the absorption of light at each wavelength. The signals representing the absorption of light over time are photoplethysmography signals or “PPG” signals. Additional information about the derivation of tissue oximetry values from emitted and detected light can be found in U.S. Pat. No. 9,861,317 (“Methods and systems for determining regional blood oxygen saturation”, Covidien LP). As used herein, the term “light” may refer to energy produced by radiative sources and may include one or more of ultrasound, radio, microwave, millimeter wave, infrared, visible, ultraviolet, gamma ray or X-ray electromagnetic radiation and any wavelength therein.

The system 100 is connected through wired or wireless connections to a network 122, such as a local area network (LAN), wide area network (WAN), or a cloud network, that couples the monitor 110 and various computing components, such as a remote server computer 124, database 126, computing device 128, and mobile device 130. Signals from the first regional oximetry sensor 112 and the second regional oximetry sensor 114 are processed by one or more processors that may be located on the monitor 110 (e.g., processor 138) or any of the distributed computing devices on the network 122. The processor or processors may derive physiological parameters from the signals. In the example of FIG. 1, the physiological parameters are first and second regional oximetry (rSO2) values from the two regional oximetry sensors 112 and 114. These values may be displayed on a display screen 132 of the monitor 110 (see e.g., rSO2 values 136, including values “66” and “55” displayed in FIG. 1). The display screen 132 may also display waveforms 134 (e.g., a plot of rSO2 values over time), alarm limits, alerts, messages, menus, and other graphic elements. While two waveforms 134 and two rSO2 values 136 represent regional oximetry data sensed by the two regional oximetry sensors 112 and 114 in FIG. 1, in other examples, only one regional oximetry sensor may be used (e.g., first regional oximetry sensor 112), producing one waveform 134 and one rSO2 value 136 measured at one tissue location of the patient. In other examples, three, four, or more regional oximetry sensors (not shown) may be used, producing three, four or more waveforms 134 and rSO2 values 136 (not shown) at various tissue locations.

As described above, monitor 110 may be coupled to or include computing components, such as the processor 138. In aspects, the processor 138 may be coupled to the regional oximetry sensors 112, 114 such that the processor 138 receives raw signals from the regional oximetry sensors 112, 114, processes those raw signals to determine physiological parameters, and outputs those parameters to the monitor 110 for display. As an example, the processor 138 may be housed in a module, preamp, puck, or block that is communicatively connected between the regional oximetry sensors 112, 114 and the monitor 110, such as through a wired or wireless connection.

In an embodiment, the physiological parameter of the patient (such as rSO2) is determined in two different ways-a baseline method and a trend method. The system 100 can operate both methods at the same time or switch between them. Each one of the two different approaches to determining the physiological parameter has benefits or advantages in different scenarios, conditions, or use cases for the caregiving team or the patient, and the system 100 has the versatility to select an appropriate approach. In an embodiment, both methods calculate the same physiological parameter, and the system 100 can select one method, switch between them, or combine them.

Examples of two methods for deriving physiological parameters will be described next. In an embodiment, the baseline method determines a baseline value (or baseline values over time) that represents an accurate absolute value of the physiological parameter being measured for most patients. However, for some patients, described further below, the baseline value is high biased with respect to the absolute value of the physiological parameter. As described above, an “absolute value” of a physiological parameter may be a derived value that is obtained indirectly (e.g., through skin) and is within an acceptable range of a true value of the physiological parameter. A “true value” of a physiological parameter may be a measured value of the physiological parameter that is obtained directly (e.g., from blood or tissue), for example.

For example, for most patients, the baseline method determines a patient's rSO2 value to within about 6 percentage points of the true value of oxygenation existing in the measured tissue, or determines a patient's SpO2 value to within about 2-4 percentage points of the true value of the oxygenation of the patient's arterial blood, or determines a patient's heart rate to within about 2-3 beats per minute of the patient's true heart rate, or the patient's core body temperature to within about 1-2 degrees Celsius or about 2-4 degrees Fahrenheit, or other examples. The amount of deviation acceptable to be considered an absolute value may vary depending on the parameter being measured, but for most patients, the baseline value is within an accepted error threshold of the true value to be considered representative of the true physiological condition of the patient and can be relied on by medical caregivers to make medical interventions. For most patients, the baseline value may also be referred to as a reference value or absolute value of the physiological parameter. For these patients, the baseline value does not need to be adjusted in order to be representative of a true value of the physiological parameter. The baseline value for these patients can be compared to physiological alarm limits, can be stored in the patient's electronic medical record (EMR), and is actionable by a medical caregiver.

The trend method determines a trend value (or trend values over time) that for most patients correlates with the accurate absolute value of the physiological parameter being measured, but is offset, displaced, or scaled from the absolute value. The trend value reflects changes or deviations in the absolute value. That is, when the absolute value changes, the trend value also changes and correctly indicates that the physiological parameter has changed and by what amount and in what direction. However, for most patients, the trend value may be offset by an amount from the absolute value and thus may fall outside of the accepted error thresholds for an accurate measurement of the physiological parameter. For example, the offset may be 3-40 percentage points for SpO2 or rSO2, or 4-8 beats per minute for heart rate, or other examples. Based on the physiological parameter, the offset may be a constant amount or a varying amount (such as a function of the physiological parameter itself). For most patients, unlike the baseline value, the trend value does not accurately represent a true value of the physiological parameter that reflects the patient's health status. Accordingly, for most patients, the trend value cannot be relied on by the medical caregivers—for example, while the trend value may cross an alarm threshold, the true value may not. However, for some patients, the baseline value is high biased with respect to the absolute value of the physiological parameter and, for these patients, the trend value more accurately represents the absolute value of the physiological parameter.

In an embodiment, the input signals used by the two methods differ. The baseline method uses a first input signal that includes the information needed to derive the baseline value. The trend method uses a second input signal that includes less or different information. For example, the first input signal may be higher-fidelity, higher-resolution, higher quality, or higher power than the second input signal. As compared to the second input signal, the first input signal may include a higher sampling rate, a larger number of data channels, greater signal power or intensity, higher resolution, less filtering, or other differences. Alternatively or in addition to those differences between the first and second input signals, the first algorithm used to derive the baseline value may be more complex than the second algorithm used to derive the trend value. For example, the first algorithm may use more processing power and time as compared to the second algorithm, and thus may be slower to provide the baseline value. The first algorithm may use a larger portion of an input signal than the second algorithm. The relatively rich information in the first input signal or used by the first algorithm enables the first method to derive the baseline value, which represents an absolute value of the physiological parameter for most patients. By comparison, the second input signal may be lower-fidelity, lower-resolution, lower quality, or lower power than the first input signal. The second input signal used by the second method may include a lower sampling rate, a smaller number of data channels, lower signal power or intensity, lower resolution, more filtering, or other differences. The second algorithm used to derive the trend value may use less processing power and less time as compared to the first algorithm, and thus may be faster to provide the trend value. The second algorithm may use a smaller portion or subset of the input signal than the first algorithm. The second input signal may be relatively more sparse or less dense, and the second method may produce a trend value that trends with the physiological parameter but does not reflect an absolute value of a physiological parameter for most patients.

In an embodiment, the characteristics of the baseline value and the trend value differ. For example, the trend value produced by the second method may have a greater responsivity (responding to changes more quickly), faster time to post, fewer drop-outs or gaps, or other differences as compared to the baseline value produced by the first method. In an embodiment, the second input signal responds to a change in the physiological parameter faster than the first input signal responds to the change.

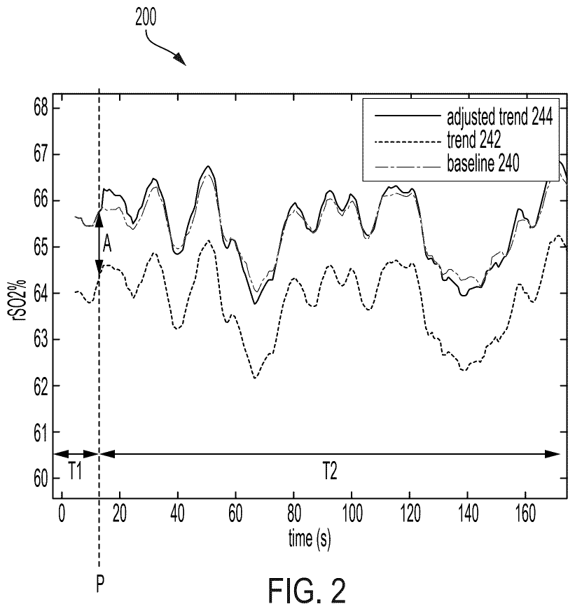

FIG. 2 depicts a plot 200 (or waveform) of baseline values and trend values of a physiological parameter over time, according to an embodiment applicable to most patients. In this example, the physiological parameter is regional oximetry (rSO2). The x-axis indicates time in seconds, and the y-axis indicates rSO2 values in percentage points. In aspects, during first time period T1, baseline values 240 may be derived using a first method based on a first input signal (e.g., including four or more wavelengths of light from a regional oximetry sensor) and trend values 242 may be derived using a second method based on a second input signal (including two of the four wavelengths). A difference (or offset) “A” may be determined between the baseline values 240 and the trend values 242 at point in time “P.” In an example, the first method may be discontinued at time point P, and the second method may be continued during the second time period T2. In this example, the trend values 242 may be adjusted by the offset A to provide an accurate and responsive estimate of the physiological parameter (e.g., rSO2) over time, as illustrated by the plot of adjusted trend values 244. In another example, the first method may be performed concurrently with the second method, as illustrated by the plot of baseline values 240 during the second time period T2. In this example, the first method may be performed periodically or continuously to provide baseline values 240 for confirmation and/or calibration the adjust trend values 244.

As illustrated by the baseline values 240 and the trend values 242, the magnitude of A is consistent across the second time period T2 of the plot 200. That is, for most patients, as the rSO2 baseline values change, the rSO2 trend values reflect the same change, but are offset by the amount A. As a result, for most patients, adjusting the rSO2 trend values by the amount A shifts the rSO2 trend values on the plot 200 to substantially match the more numerically accurate rSO2 baseline values. As illustrated by comparing the adjusted trend values 244 to the baseline values 240 of the plot 200 in FIG. 2, the adjusted trend values 244 have very good alignment with the baseline values 240 over the second time period T2. Accordingly, the adjusted trend values 244 provide good correspondence with the absolute value of the physiological parameter, within an acceptable range of the true value of the physiological parameter, which can be relied upon by medical professionals to determine a health status of the patient.

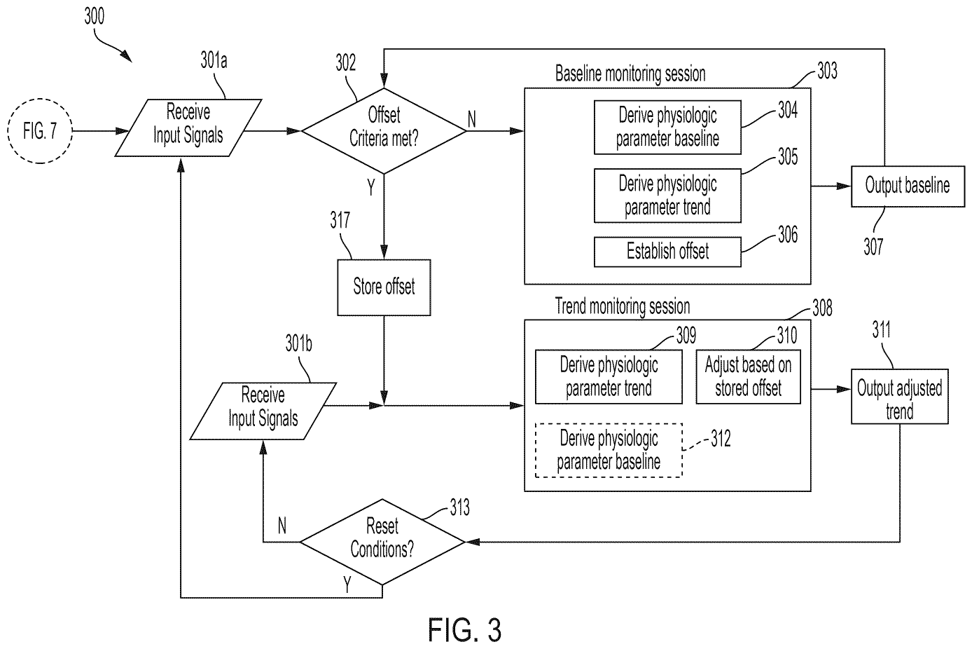

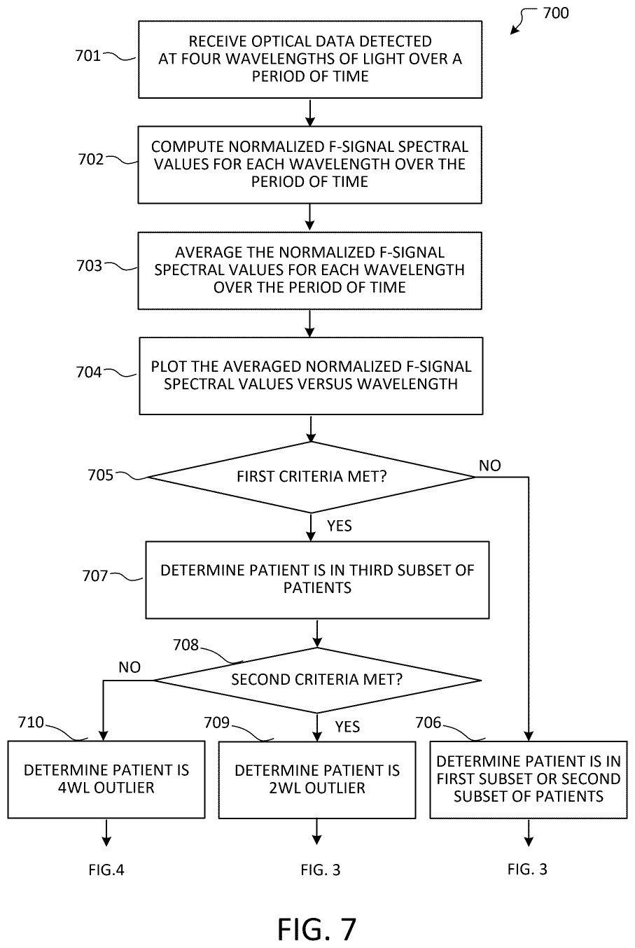

A method 300 for physiological parameter monitoring according to an embodiment is shown in FIG. 3. In some examples, prior to performing the method 300 of FIG. 3, the method 700 depicted in FIG. 7 may optionally be performed. In aspects, the method 700 determines whether the patient is a good candidate for the method 300 of FIG. 3. If method 700 determines that the patient is not a good candidate for method 300, method 300 may not be performed. Alternatively, if method 700 determines (or previously determined) that the patient is a good candidate for method 300, the method 300 may be initiated at operation 301a.

The method 300 includes operating a monitor (e.g., monitor 110) in a baseline monitoring session and a trend monitoring session and switching between them. During the baseline monitoring session, the monitor operates a first method for deriving a physiological parameter baseline value. During a trend monitoring session, the monitor operates a second method for deriving a physiological parameter trend value. The system checks respective criteria or conditions to move between the two monitoring methods, which will be described in more detail in the following paragraphs.

As shown in FIG. 3, the method 300 begins at operation 301a where it receives first input signals and second input signals from one or more physiological sensors applied to a patient. At operation 302, the system evaluates whether offset criteria or criterion have been met. If the offset criteria are not satisfied, the method 300 performs or continues to perform a baseline monitoring session at first method 303. In the baseline monitoring session, the first method 303 includes deriving a physiological parameter baseline value (e.g., “baseline value”) at operation 304 and deriving a physiological parameter trend value (e.g., “trend value”) at operation 305. In aspects, the baseline value and/or the trend value are derived using different measurement methods and/or are applied to different input signals.

An example will be described with rSO2 as the physiological parameter. In this case, the input signals are received from one or more regional oximetry sensors (e.g., first regional oximetry sensor 112 and/or second regional oximetry sensor 114 of FIG. 1) in contact with the patient's skin. The regional oximetry sensor emits light (e.g., via emitter 116 of FIG. 1) into the patient's tissue and generates signals from one or more detectors (e.g., first detector 118 and/or second detector 120 of FIG. 1) in response to the emitted light. In an embodiment, the regional oximetry sensor emits four wavelengths of light, producing one or more detector signals with four channels of data. At operation 304, all four channels (e.g., light detected by the regional oximetry sensor at all four wavelengths) are used as the first input signal to derive the physiological parameter baseline value. In this example, the baseline value is rSO2 derived from four wavelengths (e.g., rSO2 4wvl 240 of FIG. 2). In further examples, the rSO2 baseline value may be output at operation 307, may be displayed on a display (such as display 132 in FIG. 1), displayed in an alert message, compared to alarm limits for alarms or alerts, sent to a network (such as the network 122) for further processing or distribution, and/or added to a patient's EMR. The rSO2 baseline value may be further filtered (such as adding it to a running average) when it is output at operation 307.

At operation 305, two of the four channels (e.g., light detected by the regional oximetry sensor at only two of the four wavelengths) are used as the second input signal to derive the physiological parameter trend value. In this example, the physiological parameter trend value is rSO2 derived from two wavelengths (e.g., rSO2 2wvl 242 of FIG. 2). That is, the second input signal for deriving the trend value may be a subset of the first input signal.

At operation 306, the baseline monitoring session (first method 303) also includes establishing an offset between the physiological parameter baseline value and the physiological parameter trend value. In an embodiment, the offset is a difference between rSO2 4wvl and rSO2 2wvl. The offset value may also be output at operation 307. This offset can then be displayed on a display (such as display 132 in FIG. 1), displayed in an alert message, compared to alarm limits for alarms or alerts, sent to a network (such as the network 122) for further processing or distribution, and/or added to a patient's EMR. The value may be further filtered (such as adding it to a running average) when it is provided for this output.

The method 300 remains in the baseline monitoring session (e.g., first method 303) until the offset criteria are satisfied at 302. The criteria are intended to determine whether the offset has been established with a sufficient degree of confidence. In an embodiment, the criteria include threshold levels of deviation or consistency. If the offset values calculated at box 306 are consistent (such as having a deviation below a threshold), the criteria at 302 are satisfied and the offset has been established. In this case, the method 300 may continue to operation 317. If the values are inconsistent (such as having a deviation above a threshold), the criteria at 302 are not yet satisfied and the offset has not been established. In this case the method 300 continues to perform first method 303 and calculates another offset value. The first method 303 may fill a buffer of offset values and determine the deviation of the offset values in the buffer, until the criteria are satisfied at operation 302. The first method 303 may add more offset samples to the buffer or reset the buffer and fill a new buffer until the buffer fills without resetting. The buffer may be a rolling window of the offset value, such as a rolling 30-second, 40-second, 50-second, 60-second, 70-second, 80-second, or 90-second window of offset values. The first method 303 may continue to fill this rolling buffer until the values in the buffer meet the stability criterion, thus establishing a stable offset between the physiological parameter baseline value and the physiological parameter trend value.

Alternate or additional criteria may be utilized at operation 302, such as a change in the baseline value being above a threshold amount (indicating that the physiological parameter is changing quickly or hasn't settled yet), a noise level in the incoming input signals is above a threshold, the presence of a flag (such as a motion, noise, or reset flag), or other checks. If these are present, then the criteria are not met and the system remains in the baseline monitoring session (e.g., first method 303). In an aspect, the criteria applied at operation 302 include one or more signal quality metrics indicative of the quality of the input signals received at 301a. Signal quality metrics quantify the quality of the incoming signals by assessing the shape, consistency, and/or noise in the input signals. Example signal quality metrics include pulse correlation, pulse shape, skewness, derivative, consistency, pulse amplitude, signal to noise ratio, percent modulation, noise estimates, internal filter weights, cumulative filter response time, and other similar metrics that indicate the amount of noise, interference, or degradation of the incoming signals. In an example, the offset criteria applied at 302 include a sum or weighted sum of signal quality metrics. When the signal quality metrics indicate a high quality signal over a window of time, the criteria are satisfied and the method moves to box 317. In another aspect, the criteria applied at box 302 include a combination of offset stability and signal quality metrics.

In aspects, when the offset buffer has been filled and determined to be stable and/or the incoming signals are determined to meet quality criteria, the system moves to store the offset at operation 317. For example, a median, a mean, or other averaged or filtered value of the offset may be stored at operation 317. The method 300 then moves into a trend monitoring session at second method 308.

In the second method 308, the system receives second input signals at operation 301b, derives a physiological parameter trend value at operation 309 from the second input signals, and adjusts this trend value by the stored offset value at operation 310, and outputs an adjusted physiological parameter value (e.g., “adjusted value”) at operation 311. In an aspect, the adjusted value is an adjusted rSO2 value (e.g., rSO2 output 244 of FIG. 2). In further aspects, the adjusted rSO2 value may be displayed on a display (such as display 132 in FIG. 1), displayed in an alert message, compared to alarm limits for alarms or alerts, sent to a network (such as the network 122) for further processing or distribution, and/or added to a patient's EMR. The second input signals received at 301b may be different from the first input signals received at 301a. For example, the signals at 301b may be a subset of the signals received at 301a, may be obtained at a different sampling rate, frequency, power level, or may be obtained through different input channels. Alternatively, the same input signals may be obtained at both boxes 301a and 301b, and then a subset or portion of those signals is passed to operation 309 to determine the trend value.

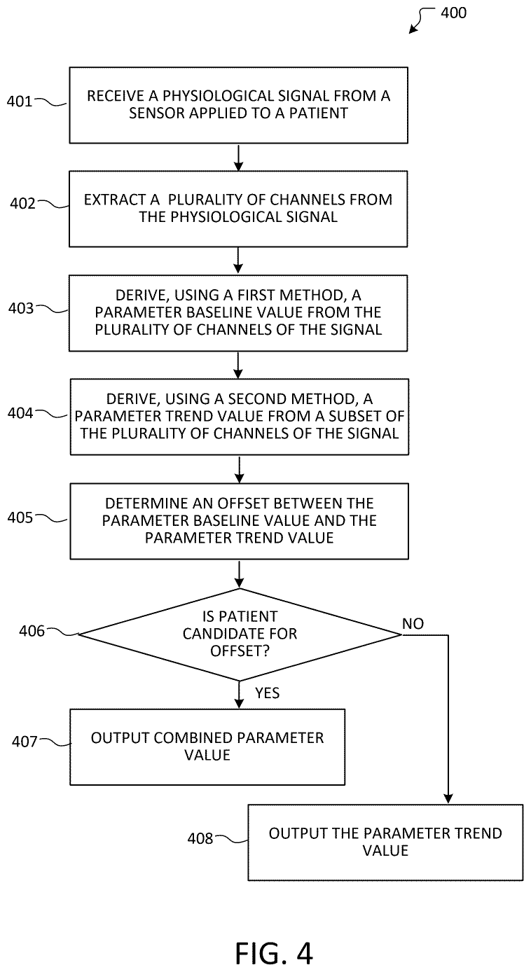

Optionally, at operation 312, the second method 308 may also include deriving the physiological parameter baseline value. In this example, the baseline value may be calculated periodically in order to re-check the offset between the baseline value and the trend value, or to average the baseline value with the adjusted value produced at operation 310. In this case, the method may include periodically obtaining the first input signals (from box 301a) and providing them into operation 312 to calculate the baseline value. The value output at operation 311 may be a combination (such as an average) of the adjusted value and the baseline value, as discussed further below with reference to FIG. 4.

In aspects, even though the second method 308 at operation 309 may derive only a trend value (e.g., not a baseline value), for most patients, the adjusted value output at operation 311 aligns with the absolute value of the physiological parameter and is actionable based on the same alarm limits and clinical practice as the absolute value. This can be accomplished during the trend monitoring session (second method 308) by using the second input signals to derive the trend value, rather than utilizing the more robust first input signals used for deriving the baseline value.

As discussed above, the second input signals may have lower resolution, lower sampling rate, lower power, lower intensity, and other characteristics different from the first input signals. As a result, the second input signals may require less power, utilize less bandwidth, and have other operating improvements and advantages than the first input signals. In an embodiment, the regional oximetry sensor consumes less power in generating the second input signals than in generating the first input signals. During the trend monitoring session, for most patients, the second method 308 benefits from these advantages while still producing an output (e.g., the adjusted value) that aligns with an absolute value of the physiological parameter. This means that the medical caregivers can rely on and interpret the adjusted value output at operation 311 without having to adjust alarm limits, take a new reference value, or change their clinical practice. In this way, the monitor operates in the same ranges of absolute values that the medical professionals are familiar with for a particular physiological parameter, including relevant medical literature, facility procedures, or clinical studies discussing or interpreting those ranges.

In an embodiment, instead of or in addition to using different input signals for deriving the baseline value and the trend value, the system uses different methods to calculate the baseline value and the trend value. Deriving the physiological parameter baseline value (at operation 304 and/or operation 312) includes using a first method 303 such as a first algorithm, machine learning model, or other approach to deriving the physiological parameter baseline value from the first input signals. Deriving the physiological parameter trend value (at operation 305 and/or operation 309) includes using a second different method 308 such as a second algorithm, machine learning model, or other approach to deriving the physiological parameter trend value from the second input signals. For example, algorithm settings in the second method 308 may be tuned differently than in the first method 303, due to the different monitoring scenario or monitoring conditions present during the trend monitoring session. Algorithm settings in the second method 308 may provide less filtering and may respond faster to changes, or different noise floor levels, ambient light sampling, criteria for applying a motion flag or determining sensor off status, and other differences. As another example, the first method 303 may employ a first machine learning model trained on the first input signals, and the second method 308 may employ a second machine learning model trained on the second input signals. These different first and second methods 303 and 308 may be re-trained, updated, or modified independently of each other.

In an embodiment, the system operates in a baseline monitoring session (e.g., first method 303) while a first set of conditions are present and operates in a trend monitoring session (e.g., second method 308) while a second, different, set of conditions are present. The first conditions may include a stable situation with high fidelity signals, little or no motion, controlled ambient light, high quality input signals, and other stable conditions. The second conditions may include a more dynamic situation with noise, interference, low quality input signals, and/or patient motion present. This could include differences in ambient light, lower fidelity signals or bandwidth, patient motion present, or other mobile or dynamic conditions. In an embodiment, the second method 308 is tailored to be more accurate than the first method 303 in the presence of these second conditions.

The method 300 includes checking reset criteria at operation 313. If the reset criteria or criterion is met, the method 300 returns to operation 301a to receive first input signals and begin a new baseline monitoring session, and if not the method 300 receives or continues to receive the second input signals at operation 301b, and returns to second method 308 (e.g., trend monitoring session). The reset criteria evaluated at operation 313 are intended to determine whether the stored offset from operation 317 remains valid. If the patient's physiological condition has changed significantly, or a large amount of time has passed, or the sensor has been removed from the patient and reapplied, or the sensor has been disconnected from the monitor, or other changes have occurred, then it may be necessary to establish a new offset. Example criteria used at operation 313 include evaluating whether the adjusted value output at operation 311 has changed by more than a threshold amount or has a rate of change higher than a threshold rate amount, detection of a sensor change (such as detecting sensor removal from the patient, sensor repositioning at a new measurement site, sensor disconnect from the monitor, sensor restart, or other sensor reset flag), evaluating whether noise levels in the input first signal or the second input signal crossed a threshold, a timer expiring, or other system checks.

Utilizing both the baseline monitoring session and the trend monitoring session can improve accuracy in physiological parameter monitoring, including for calculations of pulse oximetry (SpO2). For pulse oximetry, an absolute value of oxygen saturation can be calculated (such as at box 304 in FIG. 3) through identification and qualification of arterial pulses in the PPG signal. This approach is the preferred modality for most patients and most clinical scenarios and is used in the baseline algorithm. However, the baseline approach can struggle to identify pulses in the PPG signal when the pulses are very small in amplitude, such as during times of poor signal quality, in very cold rooms, or with very small neonatal patients. In that case, a trend value (such as the value calculated at box 309 in FIG. 3) can provide a more accurate estimate of SpO2. In an example, a trend value of SpO2 is calculated based on the DC (non-modulating) portion of the PPG signal. An output SpO2 value is then calculated based on a historic or baseline value of SpO2 for the patient adjusted by the trend.

The PPG signal consists of a modulating portion (due to the changes in light absorption caused by the arterial pulse) and a non-modulating portion (due to the light absorption caused by non-pulsatile tissue). The modulating portion of the PPG signal may be referred to as the “AC” portion, and the non-modulating portion of the PPG signal may be referred to as the “DC” portion of the signal. In scenarios where the arterial pulse is very weak or small, oxygen saturation can be estimated from the DC portion of the PPG signal. Oxygenated hemoglobin absorbs relatively more infrared light (such as wavelengths at about 900 nm) than deoxygenated hemoglobin, which absorbs relatively more red light (such as wavelengths at about 660 nm). Therefore, the DC portion of the PPG is responsive to changes in oxygenation, although it may not provide an absolute value of SpO2. In particular, one useful metric is the difference in light intensity values of the red and infrared signals in the DC portion of the PPG signal. This difference value changes in proportion to the patient's SpO2 and can be used in the trend monitoring session.

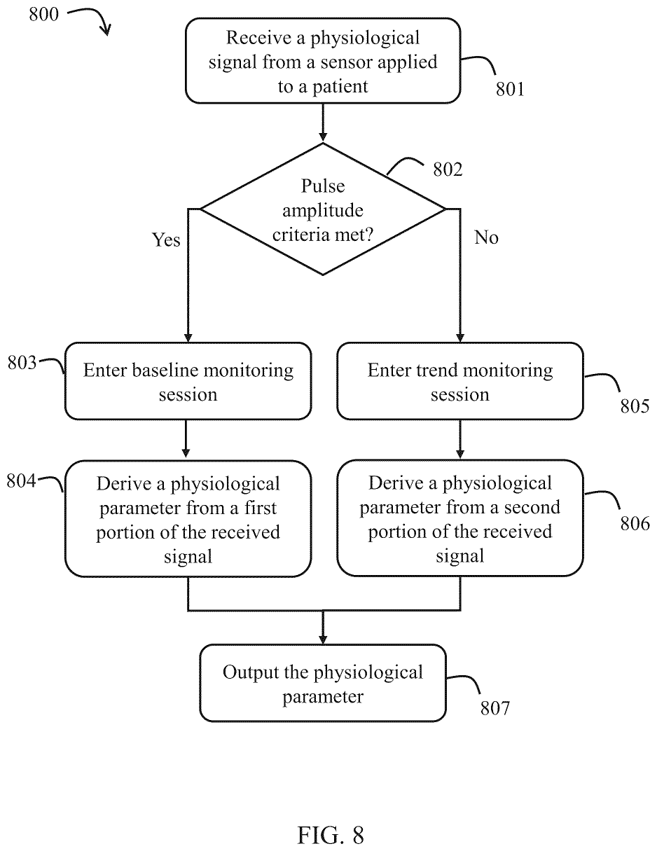

An example of such a method is shown in FIG. 8, which depicts a method 800 for calculating a physiological parameter such as SpO2. The method 800 includes receiving a physiological signal from a sensor applied to a patient, at 801. An example is a PPG signal received from a pulse oximetry sensor applied to a patient. The method then includes determining if pulse amplitude criteria or criterion is met, at 802. This check determines whether physiological pulses are present in the received signal with a sufficient amplitude for the baseline monitoring session. The determination at box 802 is based on pulse amplitude, pulse qualification, signal quality, signal strength, or other similar metrics that quantify the presence of a physiological pulse in the signal. If physiological pulses are present with a sufficient amplitude or strength, then the criteria or criterion is met. If the pulses are very small, such as below an amplitude threshold, the criteria or criterion is not met.

If the criteria or criterion is met, the method proceeds to enter a baseline monitoring session at 803. The baseline monitoring session proceeds to derive a physiological parameter from a first portion of the received signal, at 804. In the case of SpO2, the first portion of the received signal is the modulating portion of the PPG signal. The baseline algorithm includes identification and qualification of arterial pulses in the modulating portion of the PPG signal, as described above, and derives a baseline value of SpO2 from this portion of the PPG signal.

If the criteria or criterion at 802 is not met, the method proceeds to enter a trend monitoring session at 805. The trend monitoring session proceeds to derive a physiological parameter from a second portion of the received signal, at 806. In the case of SpO2, the second portion of the received signal is the non-modulating (DC) portion of the PPG signal. For example, the trend algorithm trends the patient's SpO2 from a historic or baseline SpO2 in proportion to a difference in intensity values between two different wavelengths in the DC portion of the PPG signal.

The method 800 proceeds to output the physiological parameter at 807, such as outputting either the trend SpO2 value (calculated at box 806) or the baseline SpO2 value (calculated at box 804) to a display, a message, a notification, a hospital record, or other output.

Particular examples of method 300 will be given now with rSO2 as the physiological parameter, and optical signals from a sensor as the first and second input signals. In an embodiment, the sensor is an rSO2 sensor that emits four wavelengths of light into the patient's tissue, and the first input signals received at operation 301a include light absorption signals at four wavelengths, detected by the rSO2 sensor. Deriving the physiological parameter baseline value at operation 304 includes inputting all four signals and using a first method 303 to calculate an rSO2 baseline value from the differential absorption of light at the four wavelengths (rSO24)). The first method 303 may be an empirical algorithm with coefficients or error terms derived from clinical data. The first method 303 may include a machine learning model trained on clinical data, such as a neural network. In addition to the offset, described below, the output at operation 307 may include the rSO2 baseline value calculated at operation 304.

Deriving the physiological parameter trend value at operation 305 includes using only two of the four wavelength signals (e.g., second input signals), and using a second method 308 to calculate an rSO2 trend value from the differential absorption of light at the two wavelengths (rSO2_{2λ})). The second method 308 may be a second empirical algorithm or a second machine learning model that is different from the first method 303.

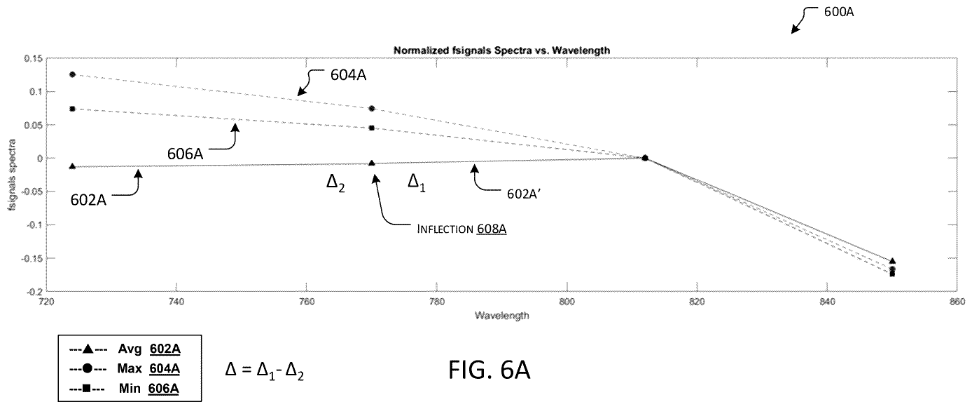

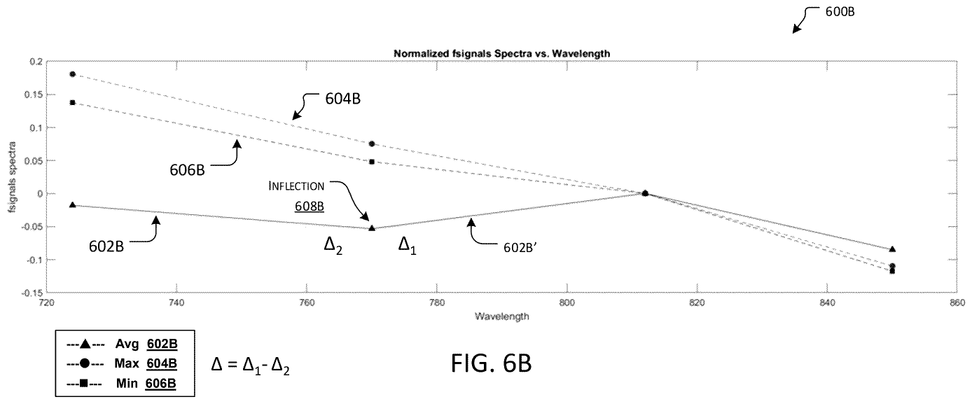

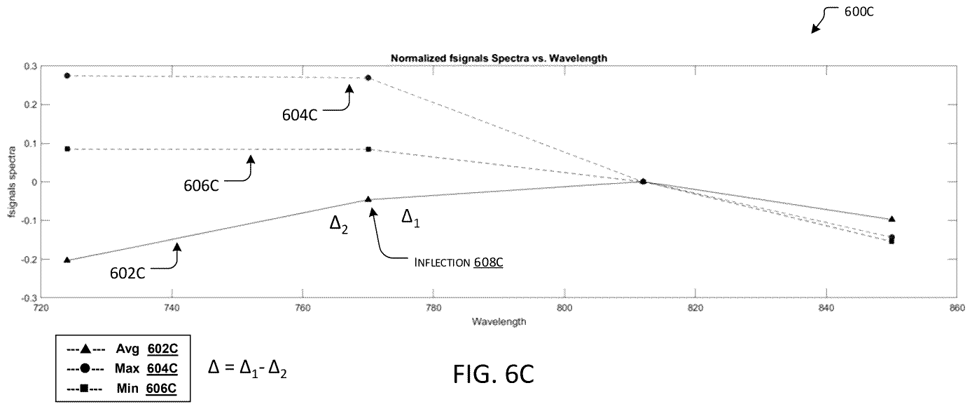

For some patients, as will be described further with respect to FIGS. 6A-7, the derived baseline and trend values of rSO2 may differ based on physiological factors that scatter or absorb light differently at different wavelengths. This can result in sensitivity to an interference source that causes wavelength dependent scattering or absorption. Thus, although the four-wavelength approach has access to more information in the form of the four input signals, for some patients, it does not necessarily result in a more accurate rSO2 baseline value over time. In this case, the two-wavelength approach may more accurately track tissue oxygen saturation over time (accuracy as compared to a reference blood gas analysis).

In an embodiment, establishing the offset at operation 306 includes filling a buffer of offset values over a time duration. For example, the first method 303 at operation 306 may include creating an initial estimate of the offset by determining a difference between the baseline value derived at operation 304 and the trend value derived at operation 305. Using rSO2 still as an example, this may include calculating a difference between the 4-wavelength baseline value and the 2-wavelength trend value, such as: Offset=rSO2_{4λ}−rSO2_{2λ}). The system may fill overlapping buffers of data as first input signals arrive from the patient and the offset is calculated at operation 306.

Once the buffer is full or a sufficient number of samples are present, the offset criteria at operation 302 are applied to determine whether the input signals have stabilized and an accurate offset value has been established between the two-wavelength rSO2 value (e.g., rSO2 trend value) and the four-wavelength rSO2 value (e.g., rSO2 baseline value). The criteria applied at operation 302 can include various ways to assess stability and confidence in the input signals, rSO2 baseline and trend calculations, and offset values. As one example, the method 300 can include checking for a change of more than a threshold amount in the baseline value over a time duration, such as checking whether the rSO2_{4λ} baseline value has changed by more than 10 percentage points within a few (e.g., 3, 4, 5, 6, 7) seconds. If so, an accurate offset may not have been established yet. This criteria is intended to mitigate large transient changes that might occur while the regional oximetry sensor is being applied. As another example, the method 300 can include assessing a deviation in the offset values in the buffer, and determining the criteria are not satisfied if the deviation is above a threshold amount.

If the criteria at operation 302 are not satisfied, the baseline monitoring session (e.g., first method 303) continues, filling up overlapping buffers of the offset value at operation 306 until the signals stabilize and the criteria at operation 302 are satisfied. Once the criteria at operation 302 are satisfied, the first method 303 stops filling the buffer and the offset is stored at operation 317. The stored offset can be a median or mean value of the offset values in the buffer, or other representative value of the offset derived from the buffered values. The method 303 then moves into the trend monitoring session (e.g., second method 308), using the stored offset to derive the rSO2 trend value from the 2-wavelength input signal (e.g., second input signal) received at operation 301b. As an example, at operation 310, the second method 308 can calculate the rSO2 adjusted value that is output at operation 311, as follows: rSO2out=rSO2_{2λ}+Offset_stored, where Offset_stored comes from the method 300 at operation 317.

Returning to FIG. 2, in an example, the baseline monitoring session is performed during time period T1 of the plot 200 and the trend monitoring session is performed during time period T2. In time period T1, the offset value A has not yet been established, and the system is calculating both the baseline value and the trend value of the physiological parameter and applying offset criteria in order to establish the offset value A. During the time period T1, the value of rSO2 that is output from the system by the first method is the baseline value calculated from the first input signals (e.g., four-wavelength). At point P on the chart, the criteria are satisfied and the offset value A is stored. Thereafter, in time period T2, the system moves into the trend monitoring session, and the adjusted value of rSO2 that is output by the second method is the trend value calculated from the second input signals (e.g., two-wavelength) adjusted by the offset value A.

In an embodiment, an initial estimate of the offset value is stored and used in an initial trend monitoring session while the baseline monitoring session continues. In this approach, the trend monitoring session (e.g., second method 308) and the baseline monitoring session (e.g., second method 303) overlap during the time period T1 when the initial estimate of the offset value has been determined but before the offset criteria at operation 302 have been satisfied. In this case, the check at operation 302 includes two different set of criteria-a first set of criteria discussed above to establish the stability of the offset value that will be stored at operation 317, as well as a second different set of criteria (initial criteria) that is used to find an initial estimate of the offset value. The purpose of this second set of criteria is to identify an initial estimate of the offset more quickly, so that the trend monitoring session can begin as soon as possible, even while a stable offset value is still being determined. The second set of criteria is satisfied more quickly than the first, such as by using a shorter buffer of offset values and/or a higher threshold for deviation. When an initial estimate of the offset value satisfies these initial criteria, the system stores the initial estimate of the offset value and initiates the trend monitoring session while continuing to operate the baseline monitoring session to establish the stable offset value at operation 306.

While both sessions are active, the system may output the adjusted value of rSO2 at operation 311, or choose between outputting the initial estimate of the offset at operation 307 and the adjusted value of rSO2 at operation 311, or combine outputs at operation 311 and operation 307 into a combined value. Once the first set of criteria at operation 302 are satisfied, the final stable value of the offset is stored at operation 317 in place of the initial estimate. In this case, the baseline monitoring session concludes, and the trend monitoring session continues with the stable offset value stored at operation 317. As mentioned above, the trend monitoring session may include optionally continuing to calculate a baseline value, which may not be output directly but used in combination with the trend value or for stability or confidence checks.

A method 400 of monitoring a patient including baseline and trend approaches is depicted in FIG. 4, according to an embodiment. The method 400 includes receiving a physiological signal from a sensor applied to a patient, at operation 401. At operation 402, a plurality of channels is extracted from the physiological signal. For example, the physiological signal may be a 4-channel light absorption signal from an rSO2 sensor. At operation 403, a physiological parameter baseline value is derived from the plurality of channels using a first method, such as deriving an rSO2 baseline value from four wavelengths of detected light. At operation 404, a physiological parameter trend value is derived from a subset of the plurality of channels, such as deriving an rSO2 trend value from two of the four wavelengths of detected light. At operation 405, an offset between the baseline value and the trend value is determined, such as a difference between the rSO2 baseline value and the rSO2 trend value.

At operation 406, it is determined whether the patient is a candidate for applying the offset. For example, a spectral analysis of one or more of the plurality of channels may be performed, as illustrated and described with reference to FIGS. 6A-6C and FIG. 7. If a spectral shape of at least one of the channels indicates that the patient is a candidate for applying the offset, the method 400 may progress to operation 407. If a spectral shape of at least one of the channels indicates that the patient is not a candidate for applying the offset, the method 400 may progress to operation 408.

At operation 407, when the patient is a candidate for applying the offset, a combined physiological parameter value is output for the patient. This combined physiological parameter value may be a combination of the trend value and the offset, or a combination of the trend value, the offset, and the baseline value. For example, the baseline value gives a first estimate of the physiological parameter, and the trend value adjusted by the offset gives a second estimate of the physiological parameter. These two estimates may be combined in a weighted average. The weights may depend on factors such as confidence in the offset, stability of the received physiological signal, noise in particular channels of the received physiological signal, and other factors. If the plurality of channels used in deriving the baseline value is noisier than the subset of channels used in deriving the trend value, for example, the weight for the baseline value in the combined average may be decreased. As another example, if the physiological parameter is changing quickly, the weight of the baseline value may be decreased and the weight of the trend value may be increased in the combined average due to the ability of the trend value to adjust to changes more quickly.

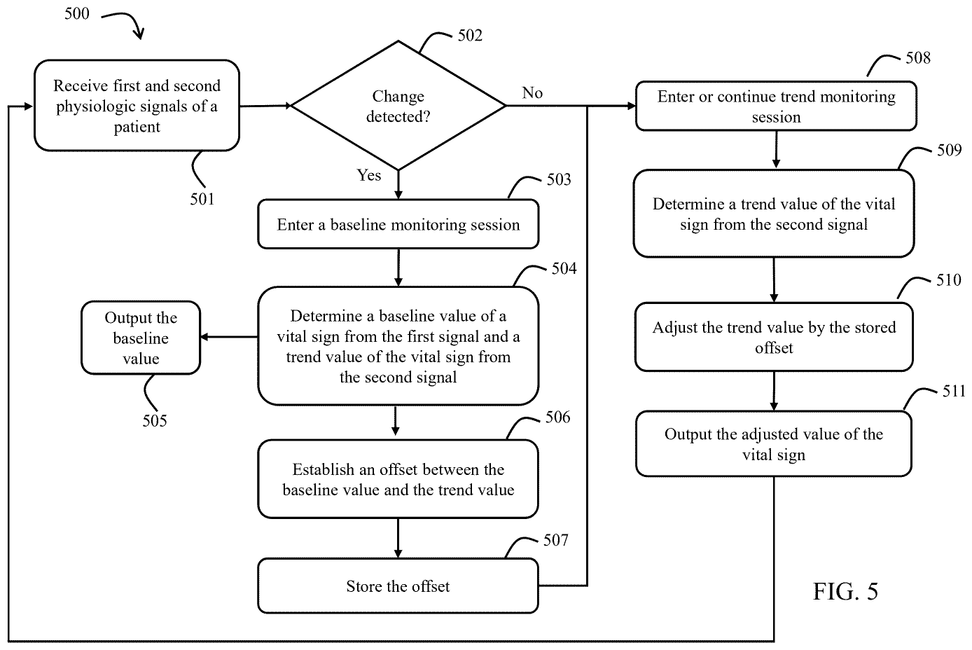

In an embodiment, when the patient is a candidate for applying the offset, the method 400 utilizes the offset (determined at operation 405) during the entire monitoring period for the patient and does not need to return to re-calculate or re-establish the offset. The received physiological input signal and algorithmic methods used for outputting the combined physiological parameter value produce a reliable output that correlates with a true physiological parameter value of the patient and does not drift over time. Thus, in this example, the offset can be relied on and combined with the trend value for the entire monitoring duration of the patient, such as 2-48 hours. In another embodiment, it may be desired to adjust the offset depending on the monitoring situation and the patient's health status, as further described with reference to FIG. 5.

At operation 408, when the patient is not a candidate for applying the offset, the parameter trend value may be output. In this case, the parameter trend value may more accurately represent the absolute value of the physiological parameter. For example, the rSO2 trend value derived from two of the four wavelengths of detected light may more accurately represent an absolute value of rSO2 that can be relied on by medical personnel.

As illustrated by FIG. 5, when the patient is a candidate for applying the offset, it may be desired to adjust the offset depending on the monitoring situation and the patient's health status. A flowchart of a method 500 for physiological parameter monitoring is shown in FIG. 5, including steps for updating the offset. The method 500 includes receiving first and second physiological signals of a patient at operation 501. The first and second physiological signals may be received from different sensors applied to the patient, and/or the second physiological signal may be a subset of information from the first physiological signal. At operation 502, the method 500 includes determining if a change is detected, such as a change in characteristics or behavior of the first and/or second physiological signals or an occurrence of one of the reset criteria described above, indicating that the offset should be re-evaluated. At operation 502, when change criteria are met (e.g., a change is detected), the method 500 enters a baseline monitoring session at operation 503.

At operation 504, the baseline monitoring session includes determining a baseline value of a vital sign from the first physiological signal and a trend value of the vital sign from the second physiological signal. At operation 505, the baseline value is output. The method 500 includes establishing an offset between the baseline value and the trend value at operation 506, such as by evaluating a stability of a difference between the baseline value and the trend value over a time period. The method 500 includes storing the offset at operation 507.

The method 500 further includes entering a trend monitoring session at operation 508, either after the offset is stored at operation 507 or when change criteria are not met (e.g., a change is not detected) at operation 502. In the trend monitoring session, the method 500 includes determining a trend value of the vital sign from the second physiological signal at operation 509, adjusting the trend value by the stored offset at operation 510, and outputting the adjusted value of the vital sign at operation 511. The method 500 then returns to the start at operation 501.

In use while monitoring a patient, the method 500 may detect a change in conditions at operation 502 and establish a new offset at operation 506 that differs from a prior offset by a large amount. This difference in offsets may occur if there is a large change in ambient conditions, signal interference, sensor performance, or other conditions that affect the offset between the trend value and the baseline value. If the difference in the offsets is large (above a threshold amount), then applying the new stored offset at operation 510 may result in a sudden step change in the adjusted value of the physiological parameter output at operation 511, even though the patient has not experienced a sudden change in physiological status. Thus, in an embodiment, storing the offset at operation 507 also includes determining whether the difference between a previous offset and a new offset is above a threshold amount. If so, then the previous offset may be shifted to the new offset over a transition period. Shifting may include employing the new offset as a function of time over the transition period. This function transitions more slowly from the previous offset to the new offset, rather than implementing an abrupt step change. The function may move incrementally (e.g., based on small discrete increments of the difference between the previous offset to the new offset) or continuously (e.g., linearly over the difference between the previous value to the new value over a time period). The period of time for the transition may be 1, 2, 3, 4, 5, or more hours, for example. In an embodiment, the period of time for the transition depends on the magnitude of the change in the offset, with the period being extended for a larger change. The rate of change of the offset during this transition period may be capped so that the change does not occur too quickly. In each case, the intention is to gradually shift from the previous offset to the new offset.

With this transition approach, the method can change the offset to maintain an accurate correlation between the trend value and the absolute value of the physiological parameter of the patient, even through changing conditions over time, and without interfering with clinical practice or workflow. In an embodiment, the operation of the system to select between methods or monitoring sessions or to update the offset is transparent to the user and does not interrupt the output of the physiological parameter.

As shown in FIG. 5, the system may switch between baseline and trend monitoring sessions, such as switching from a first baseline monitoring session into a first trend monitoring session, and then switching into a second baseline monitoring session upon identifying that the adjusted trend output by the first trend monitoring session does not adequately reflect the absolute value of the physiological parameter, for example.

FIGS. 6A-6C are charts depicting plots of normalized f-signal spectra versus wavelength for a population of patients, according to an embodiment.

As described above, a baseline value of a physiological parameter may be derived from a first input signal using a first method, and a trend value of the physiological parameter may be derived from a second input using a second method. The first input signal and the second input signal may be received from one or more sensors positioned on the skin of a patient, such as one or more regional oximetry sensors. For example, the first input signal may include data detected at four wavelengths of light and the second input signal may include data detected at two of the four wavelengths of light. In particular, a baseline value of rSO2 is derived from four wavelengths using the first method (e.g., rSO2 4wvl 240 of FIG. 2) and a trend value of rSO2 is derived from two of the four wavelengths using the second method (e.g., rSO2 2wvl 242 of FIG. 2). For most patients, the baseline rSO2 value is a more accurate representation of a true rSO2 value than the trend rSO2 value. However, for about 10% of patients, the baseline rSO2 value is high biased with respect to the true rSO2 value. For these patients, the trend rSO2 value may more accurately represent the true rSO2 value.

Unfortunately, this subset of patients (“four-wavelength (4WL) outlier patients”) is not readily identifiable based on traditional commonalities, such as observable physiological attributes (e.g., skin pigmentation), demographics, ethnicity, optical features or markers, unique sensor response, manufacturing variations, or the like. However, a spectral analysis of optical data obtained from a population of patients enables consistent identification of the 4WL outlier patients from among the population of patients. As will be described further herein, it has been discovered that the 4WL outlier patients exhibit a unique spectral shape associated with a certain band of light. In particular, the outlier patients exhibit a unique spectral shape associated with a normalized f-signal “spectra” associated with a wavelength band in a range between about 750-790 nanometers (nm), or between about 760-780 nm, or between about 765-775 nm, or any combination of ranges thereof.

The spectral analysis of the population of patients may be performed over a plurality of wavelengths (or wavelength bands). For example, the spectral analysis can be performed on optical data detected at four wavelengths of light across the population of patients. As used herein, the four wavelengths of light of the first signal may fall within a range between about 700-875 nm, or between about 710-865 nm, or between about 715-860 nm, or between about 720-855 nm, or any combination of wavelengths or ranges thereof. For example, a first wavelength (WL1) of the four wavelengths of light may fall within a range between about 700-750 nm, or between about 710-740 nm, or between about 720-730 nm, or any combination of ranges thereof. A second wavelength (WL2) of the four wavelengths of light may fall within a range between about 751-790 nm, or between about 760-780 nm, or between about 765-775 nm, or any combination of ranges thereof. A third wavelength (WL3) of the four wavelengths of light may fall within a range between about 791-830 nm, or between about 800-820 nm, or between about 805-815 nm, or any combination of ranges thereof. A fourth wavelength (WL4) of the four wavelengths of light may fall within a range between about 831-875 nm, or between about 840-865 nm, or between about 845-855 nm, or any combination of ranges thereof.

In aspects, the optical data detected at the four wavelengths of light may be obtained from different sources. In some examples, the optical data detected at the four wavelengths of light for the population of patients may be received from one or more regional oximetry sensors applied to the skin of each patient over a period of time. In this example, the “first signal,” as described herein, may be evaluated for identification of the 4WL outlier patients. In additional or alternative examples, the optical data detected at the four wavelengths of light for the population of patients may be obtained by directly testing the blood of each patient over a period of time. In aspects, the “period of time” may correspond to any amount of time suitable for obtaining stable optical data detected at the four wavelengths, e.g., from a few minutes to the full period over which the patient is monitored. In some aspects, the period of time may encompass one or more events, conditions, or activities of the patient. For example, the period of time may encompass one or more “breathe-down” events, which are defined as breathing trials during which subjects are asked to breath a specific gas mixture (e.g., O2, N2, CO2) or to complete a series of steps with incremental reductions/increases of SpO2 within target ranges.

Based on the optical data detected at the four wavelengths, one or more normalized f-signal spectra (“norm f-signal”) values may be computed for each patient over the period of time. In aspects, the f-signals at each wavelength may be normalized to one of the four wavelengths. For example, the f-signals at each wavelength may be normalized to the third wavelength (WL3), as follows:

$$Norm f - signal ( WL 1 ) = ( fdeep ( WL 1 ) - 0.75 * fshall ( WL 1 ) ) - ( fdeep ( WL 3 ) - 0.75 * fshall ( WL 3 ) )$$

$$Norm f - signal ( WL 2 ) = ( fdeep ( WL 2 ) - 0.75 * fshall ( WL 2 ) ) - ( fdeep ( WL 3 ) - 0.75 * fshall ( WL 3 ) )$$

$$Norm f - signal ( WL 3 ) = ( fdeep ( WL 3 ) - 0.75 * fshall ( WL 3 ) ) - ( fdeep ( WL 3 ) - 0.75 * fshall ( WL 3 ) )$$

$$Norm f - signal ( WL 4 ) = ( fdeep ( WL 4 ) - 0.75 * fshall ( WL 4 ) ) - ( fdeep ( WL 3 ) - 0.75 * fshall ( WL 3 ) )$$

In some examples, an average normalized f-signal (“avg f-signal”) is computed for each wavelength over the period of time. In additional or alternative examples, a maximum normalized f-signal (“max f-signal”) and a minimum normalized f-signal (“min f-signal”) are computed for each wavelength over at least a portion of the period of time. For example, the max f-signal and the min f-signal may be determined for a portion of the period of time after one or more breathe-downs are completed.

FIGS. 6A-6C illustrate plots of avg f-signal, max f-signal, and min f-signal versus wavelength for a population of patients. In particular, FIG. 6A illustrates a first plot 600A of avg f-signal, max f-signal, and min f-signal versus wavelength for a first subset of the population of patients. FIG. 6B illustrates a second plot 600B of avg f-signal, max f-signal, and min f-signal versus wavelength for a second subset of the population of patients. FIG. 6C illustrates a third plot 600C of avg f-signal, max f-signal, and min f-signal versus wavelength for a third subset of the population of patients.

As illustrated by FIG. 6A, the first plot 600A illustrates avg f-signal 602A versus wavelength, max f-signal 604A versus wavelength, and min f-signal 606A versus wavelength for the first subset of patients. As illustrated, the spectral shape of avg f-signal 602A versus wavelength is relatively linear between wavelengths of about 723 and about 815 nm. Accordingly, a plot of avg f-signal 602A for the first subset of patients is about the same or similar on either side of inflection 608A. In some examples, there may not be an inflection in the plot of avg f-signal 602A for the first subset of patients. In other examples, when there is a slight inflection 608A, according to one test, the change in avg f-signal 602A before and after inflection 608A may be represented as the difference (A) between avg f-signal 602A′ after inflection 608A (e.g., Δ1) and avg f-signal 602A before inflection 608A (e.g., Δ2), that is Δ=Δ₁−Δ₂. Here, since there is minimal if any change in avg f-signal 602A before and after inflection 608A, the difference between avg f-signal 602A′ (Δ₁) and avg f-signal (e.g., Δ₂) is equal to about zero, e.g., Δ=Δ₁−Δ₂=~0. According to another test, a change in one wavelength relative to one or more of the other wavelengths may be evaluated. For example, a spectral change associated with the third wavelength relative to the first and fourth wavelengths may be determined as follows: Δ(WL3)=(spectra (WL4)−spectra (WL3))—(spectra (WL3)−spectra (WL1))>about −0.1 and ≤about 0.1. In other aspects, the spectral change may be evaluated against any suitable threshold or range, e.g., greater than about −0.15 and less than or equal to about 0.15. In aspects, either or both tests (collectively “first criteria”) can be used to identify patients in the first subset of patients.

As illustrated by FIG. 6B, the second plot 600B illustrates avg f-signal 602B versus wavelength, max f-signal 604B versus wavelength, and min f-signal 606B versus wavelength for the second subset of patients. In aspects, the spectral shape of avg f-signal 602B exhibits a change between wavelengths of about 765 and about 775 nm. In particular, the plot of avg f-signal 602B for the second subset of patients exhibits a negative change from about 723 nm to inflection 608B and transitions to a positive change from inflection 608B to about 815 nm. Similarly to above, according one test, the change in avg f-signal 602B before and after inflection 608B may be represented as the difference (Δ) between avg f-signal 602B′ after inflection 608B (e.g., Δ₁) and avg f-signal 602B before inflection 608B (e.g., Δ₂), that is Δ=Δ₁−Δ₂. Here, the difference between the positive change in avg f-signal 602B′ after inflection 608B and the negative change in avg f-signal 602B before inflection 608B are combined to reflect the overall difference. Thus, for the second subset of patients, there is a relatively significant difference (e.g., positive change) in avg f-signal 602B before and after inflection 608B. that is Δ=Δ₁−(−Δ₂)=+Δ. According to another test, a spectral change associated with the third wavelength relative to the first and fourth wavelengths may be determined as follows: Δ(WL3)=(spectra (WL4)−spectra (WL3))−(spectra (WL3)−spectra (WL1))>about 0.1. In other aspects, the spectral change may be evaluated against another suitable threshold or range, e.g., greater than about 0.05 to about 0.15, or any threshold or range therebetween. In aspects, either or both tests (collectively “first criteria”) can be used to identify patients in the second subset of patients.

As illustrated by FIG. 6C, the third plot 600C illustrates avg f-signal 602C versus wavelength, max f-signal 604C versus wavelength, and min f-signal 606C versus wavelength for the third subset of patients. In aspects, the spectral shape of avg f-signal 602C exhibits a change between wavelengths of about 765 and about 775 nm. In particular, the plot of avg f-signal 602C for the third subset of patients exhibits a positive change from about 723 nm to inflection 608C and transitions to a less positive change from inflection 608C to about 815 nm. Similarly to above, according to one test, the change in avg f-signal 602C before and after inflection 608C may be represented as the difference (Δ) between avg f-signal 602C′ after inflection 608C (e.g., Δ₁) and avg f-signal 602C before inflection 608C (e.g., Δ₂), that is Δ=Δ₁−Δ₂. Here, the difference between the less positive change in avg f-signal 602C′ after inflection 608C and the more positive change in avg f-signal 602C before inflection 608C are offset to reflect the overall change. Thus, for the third subset of patients, there is a negative change in avg f-signal 602C before and after inflection 608C. that is Δ=Δ₁−Δ₂=−Δ. According to another test, a spectral change associated with the third wavelength relative to the first and fourth wavelengths may be determined as follows: Δ(WL3)=(spectra (WL4)−spectra (WL3))−(spectra (WL3)−spectra (WL1))≤about −0.1. In other aspects, the spectral change may be evaluated against another suitable threshold or range, e.g., less than or equal to about −0.05 to about −0.15, or any threshold or range therebetween. In aspects, either or both tests (collectively “first criteria”) can be used to identify patients in the third subset of patients.

In aspects, for patients in the first subset and the second subset of patients, the methods and systems disclosed herein perform well. That is, for patients in the first and second subset of patients, applying an offset to a trend value of a physiological parameter (e.g., adjusted trend rSO2 value) results in an accurate representation of the absolute or true value of the physiological parameter (e.g., true rSO2 value). However, for some patients in the third subset of patients (e.g., ΔWL outlier patients), applying the offset to the trend value results in a high biased representation of the absolute or true value of the physiological parameter. Alternatively, for other patients in the third subset of patients (e.g., “two-wavelength (2WL) outlier patients”), the trend value derived using two wavelengths of light is low biased. Thus, for the 2WL patients, applying the offset to the trend value may result in a more accurate representation of the absolute or true value of the physiological parameter.

Accordingly, once the third subset of patients is identified using spectral analysis, the 4WL outlier patients, for whom the offset should not be applied, are distinguished from the 2WL outlier patients, for whom the offset should be applied. In some examples, the 2WL outlier patients may be identified based on further spectral analysis. For example, second criteria can be applied to the averaged normalized f-signal spectra values for patients identified within the third subset of patients. In aspects, the second criteria may include determining a spectral change in the first wavelength relative to the third wavelength as follows: Δ(WL1)=spectra (WL3)−spectra (WL1)≤about −0.2. In other aspects, the spectral change in the first wavelength relative to the third wavelength may be evaluated against another suitable threshold or range, e.g., less than or equal to about −0.15 to about −0.25, or any threshold or range therebetween.

FIG. 7 is a flowchart depicting a method of determining whether to apply an offset when deriving a physiological parameter, according to an embodiment. As described above, for most patients, the methods and systems disclosed herein output an accurate representation of the absolute or true value of a physiological parameter by applying an offset to a trend value of the physiological parameter. However, for some patients (e.g., ΔWL outlier patients), applying the offset to the trend value results in a high biased representation of the absolute or true value of the physiological parameter. Method 700 applies criteria for identifying patients who are and who are not good candidates for determining and applying the offset according to method 300 of FIG. 3.

Method 700 begins with operation 701, where optical data for the patient is received. For example, the optical data may be received based on drawing blood from the patient or by applying regional oximetry sensors to the patient, for example. In aspects, the optical data for the patient is detected at four wavelengths of light over a period of time. The “period of time” may correspond to any amount of time suitable for obtaining stable optical data detected at the four wavelengths of light, e.g., 5, 6, 7, 8, 9, 10 minutes.

At operation 702, based on the optical data, one or more normalized f-signal spectra values may be computed over the period of time. In aspects, the f-signals at each wavelength may be normalized to one of the four wavelengths. For example, the f-signals at each wavelength may be normalized to the third wavelength (WL3), as follows:

$$Norm f - signal ( WL 1 ) = ( fdeep ( WL 1 ) - 0.75 * fshall ( WL 1 ) ) - ( fdeep ( WL 3 ) - 0.75 * fshall ( WL 3 ) )$$

$$Norm f - signal ( WL 2 ) = ( fdeep ( WL 2 ) - 0.75 * fshall ( WL 2 ) ) - ( fdeep ( WL 3 ) - 0.75 * fshall ( WL 3 ) )$$

$$Norm f - signal ( WL 3 ) = ( fdeep ( WL 3 ) - 0.75 * fshall ( WL 3 ) ) - ( fdeep ( WL 3 ) - 0.75 * fshall ( WL 3 ) )$$

$$Norm f - signal ( WL 4 ) = ( fdeep ( WL 4 ) - 0.75 * fshall ( WL 4 ) ) - ( fdeep ( WL 3 ) - 0.75 * fshall ( WL 3 ) )$$

At operation 703, an average of the normalized f-signal spectra values for each wavelength may be computed over the period of time and, at operation 704, the averaged normalized f-signal spectra values may be plotted versus wavelength. In aspects, an inflection may be detected in the plotted normalized f-signals. In some aspects, the inflection may occur between about 765-775 nm, or between about 767-773 nm; in other aspects, the inflection may occur at a wavelength greater than or equal to about 770 nm.

At operation 705, first criteria are applied to the plotted spectral data. For example, according to one test, a difference (Δ) between the average f-signal spectra values after an inflection (e.g., Δ₁) and the average f-signal spectra values before the inflection (e.g., Δ₂) may be determined, that is Δ=Δ₁−Δ₂. In aspects, if the change is less than zero, the first criteria are met; and if the change is greater than or equal to zero, the first criteria are not met. According to another test, a change in one wavelength relative to one or more of the other wavelengths may be evaluated. For example, a spectral change associated with the third wavelength relative to the first and fourth wavelengths may be determined as follows: Δ(WL3)=(spectra (WL4)−spectra (WL3))−(spectra (WL3)−spectra (WL1)). In aspects, if the change is less than or equal to about −0.1, the first criteria are met; and if the change is greater than about −0.1, the first criteria are not met. In other aspects, the spectral change may be evaluated against another suitable threshold or range, e.g., less than or equal to about −0.05 to −0.15, or any threshold or range therebetween. In aspects, either or both tests (collectively “first criteria”) can be used to identify patients who are not candidates for the method 300 of FIG. 3. If the first criteria are met, the method progresses to operation 707. If the first criteria are not met, the method progresses to operation 706.

At operation 706, when the first criteria are not met, it may be determined that the patient is within one of a first subset or a second subset of patients, as described above with reference to FIGS. 6A-6B. Patients in the first subset and the second subset of patients are good candidates for the method 300 of FIG. 3. In this case, in response to determining that the patient is within one of the first subset or the second subset of patients, the method 300 of FIG. 3 may be performed.

At operation 707, when the first criteria are met, it may be determined that the patient is within a third subset of patients, as described above with reference to FIG. 6C.

At operation 708, second criteria are applied to the plotted spectral data. For example, the second criteria may include determining a spectral change in the first wavelength relative to the third wavelength as follows: Δ(WL1)=spectra (WL3)−spectra (WL1). In aspects, if the spectral change is less than or equal to about −0.2, the second criteria are met; and if the spectral change is greater than about −0.2, the second criteria are not met. In other aspects, the spectral change may be evaluated against another suitable threshold or range, e.g., less than or equal to about −0.15 to about −0.25, or any threshold or range therebetween. If the second criteria are met, the method progresses to operation 709. If the second criteria are not met, the method progresses to operation 710.

At operation 709, when the second criteria are met, it may be determined that the patient is a 2WL outlier patient. 2WL outlier patients are good candidates for the method 300 of FIG. 3. In this case, in response to determining that the patient is a 2WL outlier patient, the method 300 of FIG. 3 may be performed.

At operation 710, when the second criteria are not met, it may be determined that the patient is a 4WL outlier patient. 4WL outlier patients are not good candidates for the method 300 of FIG. 3. In this case, in response to determining that the patient is a 4WL outlier patient, the method 400 of FIG. 4 may be performed. In this case, with reference to FIG. 4, it may be determined that the patient is not a candidate for the offset at operation 406, and the parameter trend value for the patient may be output at operation 408.

There are various options for outputting the physiological parameter value (whether the baseline value during a baseline monitoring session or a trend value, an adjusted trend value or combined trend value during a trend monitoring session). The value may be output to a display screen such as the display 132 in FIG. 1, or to a display screen on a remote device over the network 122. Outputting can include sending messages to medical caregivers through the network 122, including alert or alarm messages (indicating a change in the physiological parameter, the sensor status, the system status, or other changes with the patient). An alarm message may be created when the output physiological parameter value crosses an alarm threshold. Alert or alarm messages may include audible or visible alarms or indicators. The output value may also be displayed as a waveform showing the value over time. The output value and historical or average or sampled values may be stored in the patient's EMR.

While a wired cable is shown in some of the figures connecting system elements, wireless connections may be used in place of wired connections. For example, the multi-site sensor may include a wireless antenna that transmits the deflection signal and physiological signal(s) to a wireless receiver of a monitor, network, or other computing device. Components of the medical monitor may be distributed over a network, with various processors, hardware memory, databases, logic, encoded information, and algorithms stored or executed at different locations or by different computing elements connected to the network. The network may be a local area network, system network, or distributed cloud network.

The following describes aspects of the disclosure herein that may be used alone or in combination.

Example 1: A method for medical monitoring of a patient, comprising: receiving, from one or more sensors applied to a patient, a first physiological signal and a second physiological signal responsive to a physiological parameter of the patient; determining, by one or more computer processors, a baseline value of the physiological parameter from the first physiological signal and a trend value of the physiological parameter from the second physiological signal; outputting the baseline value during a baseline monitoring session; establishing an offset between the baseline value and the trend value during the baseline monitoring session; storing the offset; switching from the baseline monitoring session to a trend monitoring session; in response to switching to the trend monitoring session, adjusting the trend value by the offset; and outputting the adjusted trend value of the physiological parameter during the trend monitoring session.

Example 2: The method of example 1, wherein the second physiological signal comprises a subset of data from the first physiological signal.

Example 3: The method of example 2, wherein the first physiological signal comprises absorption data from four different wavelengths emitted by the one or more sensors, and wherein the second physiological signal comprises absorption data from two of the four wavelengths.

Example 4: The method of one of examples 1-3, wherein establishing the offset comprises determining that the first physiologic signal satisfies a signal quality criterion.

Example 5: The method of example 1-3, wherein establishing the offset comprises determining that a difference between the baseline value and the trend value satisfies a stability criterion.

Example 6: The method of one of examples 1-5, wherein the baseline monitoring session comprises a first baseline monitoring session and the offset comprises a first offset, and wherein the method further comprises switching into a second baseline monitoring session upon identifying a change in the first offset during the trend monitoring session.

Example 7: The method of example 6, wherein the second baseline monitoring session comprises establishing a second offset and gradually shifting from the first offset to the second offset.

Example 8: The method of one of examples 1-7, wherein the second physiological signal consumes less power from the one or more sensors than the first physiological signal.

Example 9: The method of one of examples 1-8, wherein the second physiological signal responds to a change in the physiological parameter faster than the first physiological signal responds to the change.

Example 10: A method for monitoring a physiological parameter of a patient, comprising: receiving a physiological signal from a sensor applied to a patient, the physiological signal comprising a plurality of channels of data responsive to a physiological parameter of the patient; deriving a physiological parameter baseline from the plurality of channels; deriving a physiological parameter trend from a subset of the plurality of channels; storing an offset between the physiological parameter baseline and the physiological parameter trend; producing a combined physiological parameter of the patient by adjusting the physiological parameter trend by the offset; and outputting the combined physiological parameter for monitoring or display.

Example 11: The method of example 10, wherein the offset comprises a first offset and wherein the method further comprises determining a change in the first offset and updating the first offset to a second offset based on the change.

Example 12: The method of one of examples 10-11, wherein updating the first offset to the second offset comprises continuously shifting from the first offset to the second offset over a time duration.

Example 13: The method of Example 10, further comprising: based on analyzing spectral data associated with the patient, determining whether the physiological parameter trend should be adjusted by the offset; and wherein the producing and outputting the combined physiologic parameter is in response to determining that the physiological parameter trend should be adjusted by the offset.

Example 14: The method of Example 13, wherein analyzing the spectral data associated with the patient further comprises: receiving optical data associated with the patient, the optical data detected at a plurality of wavelengths; computing the spectral data for each wavelength of the plurality of wavelengths; plotting the spectral data versus wavelength; and applying criteria to the plotted spectral data to determine whether the physiological parameter trend should be adjusted by the offset, wherein when the physiological parameter trend should not be adjusted by the offset, the method further comprising: outputting the physiological parameter trend for monitoring or display.

Example 15: The method of one of examples 10-14, wherein the sensor comprises an oximetry sensor and the plurality of channels of data comprises four channels, each channel comprising data from one of four different wavelengths emitted by the oximetry sensor, and wherein the physiological parameter baseline is derived from the four channels, and wherein the physiological parameter trend is derived from two of the four channels.

Example 16: A method for determining whether to apply an offset to a physiological parameter trend value for a patient, the method comprising: receiving optical data associated with the patient, the optical data being detected at a plurality of wavelengths over time; computing f-signal spectra values for each wavelength of the plurality of wavelengths over time; applying first criteria to the computed f-signal spectra values; when the first criteria are met, applying second criteria to the computed f-signal spectra values; and when the second criteria are not met, outputting the physiological parameter trend value.

Example 17: The method of example 16, further comprising: normalizing the f-signal spectra values with respect to one wavelength of the plurality of wavelengths over time; plotting the normalized f-signal spectra values versus wavelength; and applying first criteria to the plotted normalized f-signal spectra values.

Example 18: The method of one of examples 16-17, wherein when the second criteria are met, the method further comprises: producing a combined physiological parameter value of the patient by applying the offset to the physiological parameter trend value; and outputting the combined physiological parameter value.

Example 19: The method of example 16, wherein applying the first criteria comprises: plotting the f-signal spectra values versus wavelength; identifying an inflection point in the plotted f-signal spectra values; and evaluating a change in the plotted f-signal spectra values on either side of the inflection point.

Example 20: The method of Example 1, wherein the first physiologic signal comprises a modulating portion of a photoplethysmography (PPG) signal, and wherein the second physiologic signal comprises a non-modulating portion of the PPG signal.

Example 21: A method for determining whether to apply an offset to a physiological parameter trend value for a patient, the method comprising: receiving optical data associated with the patient, the optical data being detected at a plurality of wavelengths over time; computing f-signal spectra values for each wavelength of the plurality of wavelengths over time; applying first criteria to the computed f-signal spectra values; when the first criteria are met, applying second criteria to the computed f-signal spectra values; and when the second criteria are not met, outputting the physiological parameter trend value.

Example 22: The method of Example 21, further comprising: normalizing the f-signal spectra values with respect to one wavelength of the plurality of wavelengths over time; plotting the normalized f-signal spectra values versus wavelength; and applying first criteria to the plotted normalized f-signal spectra values.

Example 23: The method of claim 21, wherein applying the first criteria comprises: plotting the computed f-signal spectra values; identifying an inflection point in the plotted f-signal spectra values; and evaluating a change in the plotted f-signal spectra values on either side of the inflection point.

Example 24: A method for monitoring a physiological parameter of a patient, comprising: receiving a physiological signal from a sensor applied to a patient, the physiological signal comprising a plurality of channels of data responsive to a physiological parameter of the patient; deriving a physiological parameter baseline from the plurality of channels; deriving a physiological parameter trend from a subset of the plurality of channels; storing an offset between the physiological parameter baseline and the physiological parameter trend; producing a combined physiological parameter of the patient by adjusting the physiological parameter trend by the offset; and outputting the combined physiological parameter for monitoring or display.

Example 25: The method of Example 24, wherein the offset comprises a first offset and wherein the method further comprises determining a change in the first offset and updating the first offset to a second offset based on the change.

Example 26: The method of Example 25, wherein updating the first offset to the second offset comprises continuously shifting from the first offset to the second offset over a time duration.

Example 27: The method of Example 26, wherein continuously shifting comprises moving linearly from the first offset to the second offset over the time duration.

Example 28: The method of Example 24, wherein producing the combined physiologic parameter of the patient further comprises averaging the adjusted physiologic parameter trend and the physiologic parameter baseline.

Example 29: A method for monitoring a physiologic parameter of a patient, comprising: receiving, at a medical monitor, a physiological signal from a sensor applied to a patient, the physiological signal being responsive to a physiologic parameter of the patient; operating the medical monitor in a first measurement mode comprising: deriving a first estimate of the physiologic parameter of the patient; deriving a second estimate of the physiologic parameter of the patient; deriving an offset between the first estimate and the second estimate; and outputting the first estimate to an alert or display; evaluating a stability of the offset; upon determining that the stability satisfies a criterion, switching to a second measurement mode comprising: deriving the second estimate of the physiologic parameter of the patient; adjusting the second estimate by the offset; and outputting the adjusted second estimate to the alert or display; subsequently, detecting a change in the stability, the second estimate, the physiological signal, or the sensor; and returning the medical monitor to the first measurement mode due to the change.

Example 30: The method of Example 29, wherein the physiologic parameter comprises tissue oxygen saturation of the patient.

Example 31: The method of Example 29, wherein the physiologic parameter comprises arterial oxygen saturation of the patient.

Example 32: The method of Example 29, wherein the physiologic parameter comprises a respiratory parameter of the patient.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A method for medical monitoring of a patient, comprising:
receiving, from one or more sensors applied to a patient, first and second physiologic signals responsive to a physiologic parameter of the patient;
determining, by one or more computer processors, a baseline value of the physiologic parameter from the first physiologic signal and a trend value of the physiologic parameter from the second physiologic signal;
outputting the baseline value during a baseline monitoring session;
establishing an offset between the baseline value and the trend value during the baseline monitoring session;
storing the offset;
switching from the baseline monitoring session to a trend monitoring session;
in response to switching to the trend monitoring session, adjusting the trend value by the offset; and
outputting the adjusted trend value of the physiologic parameter during the trend monitoring session.

2. The method of claim 1, wherein the second physiologic signal comprises a subset of data from the first physiologic signal.

3. The method of claim 2, wherein the first physiologic signal comprises absorption data from four different wavelengths emitted by the one or more sensors, and wherein the second physiologic signal comprises absorption data from two of the four wavelengths.

4. The method of claim 1, wherein establishing the offset comprises determining that the first physiologic signal satisfies a signal quality criterion.

5. The method of claim 1, wherein establishing the offset comprises determining that a difference between the baseline value and the trend value satisfies a stability criterion.

6. The method of claim 1, wherein the baseline monitoring session comprises a first baseline monitoring session and the offset comprises a first offset, and wherein the method further comprises switching into a second baseline monitoring session upon identifying a change in the first offset during the trend monitoring session.

7. The method of claim 6, wherein the second baseline monitoring session comprises establishing a second offset and gradually shifting from the first offset to the second offset.

8. The method of claim 1, wherein the second physiologic signal consumes less power from the one or more sensors than the first physiologic signal.

9. The method of claim 1, wherein the second physiologic signal responds to a change in the physiologic parameter faster than the first physiologic signal responds to the change.

10. A method for monitoring a physiologic parameter of a patient, comprising:
receiving a physiological signal from a sensor applied to a patient, the physiologic signal comprising a plurality of channels of data responsive to a physiologic parameter of the patient;
deriving a physiologic parameter baseline from the plurality of channels;
deriving a physiologic parameter trend from a subset of the plurality of channels;
storing an offset between the physiologic parameter baseline and the physiologic parameter trend;
producing a combined physiologic parameter of the patient by adjusting the physiologic parameter trend by the offset; and
outputting the combined physiologic parameter for monitoring or display.

11. The method of claim 10, wherein the offset comprises a first offset and wherein the method further comprises determining a change in the first offset and updating the first offset to a second offset based on the change.

12. The method of claim 11, wherein updating the first offset to the second offset comprises continuously shifting from the first offset to the second offset over a time duration.

13. The method of claim 10, further comprising:
based on analyzing spectral data associated with the patient, determining whether the physiological parameter trend should be adjusted by the offset; and
wherein the producing and outputting the combined physiologic parameter is in response to determining that the physiological parameter trend should be adjusted by the offset.

14. The method of claim 13, wherein analyzing the spectral data associated with the patient further comprises: receiving optical data associated with the patient, the optical data detected at a plurality of wavelengths; computing the spectral data for each wavelength of the plurality of wavelengths; plotting the spectral data versus wavelength; and applying criteria to the plotted spectral data to determine whether the physiological parameter trend should be adjusted by the offset, wherein when the physiological parameter trend should not be adjusted by the offset, the method further comprising: outputting the physiological parameter trend for monitoring or display.

15. The method of claim 10, wherein the sensor comprises an oximetry sensor and the plurality of channels of data comprises four channels, each channel comprising data from one of four different wavelengths emitted by the oximetry sensor, and
wherein the physiologic parameter baseline is derived from the four channels, and
wherein the physiologic parameter trend is derived from two of the four channels.

16. A method for monitoring a physiologic parameter of a patient, comprising:
receiving, at a medical monitor, a physiological signal from a sensor applied to a patient, the physiological signal being responsive to a physiologic parameter of the patient;
operating the medical monitor in a first measurement mode comprising:
deriving a first estimate of the physiologic parameter of the patient;
deriving a second estimate of the physiologic parameter of the patient;
deriving an offset between the first estimate and the second estimate; and
outputting the first estimate to an alert or display;
evaluating a stability of the offset;
upon determining that the stability satisfies a criterion, switching to a second measurement mode comprising:
deriving the second estimate of the physiologic parameter of the patient;
adjusting the second estimate by the offset; and
outputting the adjusted second estimate to the alert or display;
subsequently, detecting a change in the stability, the second estimate, the physiological signal, or the sensor; and
returning the medical monitor to the first measurement mode due to the change.

17. The method of claim 16, wherein the physiologic parameter comprises tissue oxygen saturation of the patient.

18. The method of claim 16, wherein the physiologic parameter comprises arterial oxygen saturation of the patient.

19. The method of claim 16, wherein the physiologic parameter comprises a respiratory parameter of the patient.

20. The method of claim 1, wherein the first physiologic signal comprises a modulating portion of a photoplethysmography (PPG) signal, and wherein the second physiologic signal comprises a non-modulating portion of the PPG signal.
